(19) United States
(12) Patent Application Publication
(10) Pub. No.: US 2003/0049258 A1
(43) Pub. Date: Mar. 13, 2003

(54) Method of increasing the contractility of a heart, a heart muscle or cells of a heart muscle

(76) Inventors: Martin Ungerer, Munchen (DE); Gotz Munch, Munchen (DE); Christine Baumgartner, Munchen (DE); Kai Rosport, Munchen (DE); Karl-Ludwig Laugwitz, Munchen (DE); Martin Lohse, Wurzburg (DE)

Correspondence Address: MYERS BIGEL SIBLEY & SAJOVEC, PO BOX 37428, RALEIGH, NC, 27627, US

(21) Appl. No.: 09/951,030

(22) Filed: Sep. 11, 2001

(51) Int. Cl. A61K 48/00; C12Q 1/00; G01N 33/53; A61K 39/395

(52) U.S. Cl. 424/146.1; 514/12; 514/44; 435/4; 435/7.1

(57) ABSTRACT

A method of increasing the contractility of a heart, a heart muscle or cells of a heart muscle i by administering an agent capable of binding to a phosducin binding site of Gβγ is provided. Said phosducin binding site is preferably a binding site of N-terminal truncated phosducin. Further, methods of identifying compounds capable of increasing the contractility of a heart muscle and methods of identifying compounds capable of inhibiting Gβγ-mediated processes are provided.

### TECHNICAL FIELD OF THE INVENTION

The present invention relates to the treatment of congestive heart failure. In particular, the present invention relates to a method of increasing the contractility of a heart, a heart muscle or cells of a heart muscle. Moreover, the present invention relates to methods of identifying compounds capable of increasing the contractility of a muscle, in particular, a heart muscle, or cells of a heart muscle. Finally, the present invention relates to novel proteins including antibodies, as well as polynucleotides and vectors useful in the methods of the present invention.

### BACKGROUND OF THE INVENTION

Congestive heart failure is a chronic disease affecting about 5 Million people in the United States. The five year-mortality rate of patients suffering from congestive heart failure is presently at a level of 50% whereby specific forms or additional complications lead to drastically increased mortality rates. Congestive heart failure develops when the heart must cope for an extended period of time with an abnormally high demand upon cardiac contractility. An abnormally high demand may be caused by cardiovascular disease such as hypertension and myocardial ischemia, cardiomyopathy or congenital heart disease.

Conventional drug therapies of congestive heart failure are directed to an increase of the cardiac output as well as relief of pulmonary congestion and peripheral edema. Cardiac output is conventionally increased by administration of positive inotropic agents stimulating myocardial contractility by enhancing the force and velocity of the myocardial contraction. The oldest and still most important drugs for the treatment of congestive heart failure are based on cardiac glycosides. Cardiac glycosides represent a class of closely related natural products acting directly on the myocardium, specifically, on the membrane-bound Na⁺ and K⁺-dependant adenosine triphosphatase. Under physiological conditions, this enzyme hydrolizes ATP to achieve the exchange of intracelluar Na⁺ for extracellular K⁺ against concentration gradients. Cardiac glycosides bind to the specific receptor site of the enzyme at the external surface of the membrane-bound enzyme. In addition, the active transport system by glycoside leads to an increase in intracellular Na⁺ and a decrease in intra-cellular K⁺. The accumulation of Na⁺ is linked to an increased influx of Ca²⁺ which is regulated by the Na⁺/Ca²⁺ carrier system. As a consequence, more Ca²⁺ is available for interaction with myofibrilles.

Cardiac glycosides show a complex set of effects including the desired positive inotropic effect and a decrease of the rate of the heart. Due to the very narrow therapeutic range of 1.5 to 2.5, therapy with cardiac glycoside is difficult. In some patients, toxic symptoms are observed at doses required for providing at least partially therapeutic effects. The toxicity of cardiac glycosides comprises both extracardial and cardial effects. Cardiac toxicity produces arrhythmias leading in severe cases to ventricular fibrilations with subsequent systolic arrest and death.

Although cardiac glycosides are able to improve the course and the symptoms of congestive heart failure, an improvement of the mortality rate has not been demonstrated with conventional positive inotropic agents.

Therefore, the development of alternative therapies for the treatment of congestive heart failure and the identification of agents having positive inotropic effects is highly desirable. The identification of agents having positive inoptropic effects is currently limited by the known biochemical mechanisms on which the contractility of the heart is based. As a consequence, the possibilities for the development of alternative therapies are also extremely limited.

Gβγ is a dimeric protein complex of G proteins which act as signal transducers of many membrane-bound receptors. G proteins are membrane-bound heterotrimeric protein complexes consisting of a GTP/GDP-binding a subunit and the tightly bound Gβγ complex. G protein mediated signaling is subject to a variety of regulatory controls. Although control is mostly exerted at the receptor level, several proteins have been described to alter the activity of G proteins by direct interaction.

Phosducin is an example for a Gβγ binding protein which regulates G protein signalling (Bauer et al., 1992; Lee et al., 1992). Phosducin is present in the retina and the pineal gland (Reig 1990). Moreover, phosducin has also been purified from brain (Bauer et al., 1992), and mRNA and protein expression have been detected in other tissues (Bauer et al., 1992; Danner and Lohse, 1996). Phosducin binding to Gβγ is known from Gaudet et al. 1996 and WO 98/040402.

### SUMMARY OF THE INVENTION

It is a problem of the invention to provide novel methods of increasing the contractility of a heart, a heart muscle or cells of a heart muscle, which are useful in therapy of congestive heart failure. Moreover, it is a problem of the invention to provide methods of identifying compounds capable of increasing the contractility of a heart, a heart muscle, or cells of a heart muscle.

The present invention provides a method of increasing the contractility of a heart, a heart muscle or cells of a heart muscle by administering an agent capable of binding to a phosducin binding site of Gβγ.

Moreover, the present invention provides a method of increasing the contractility of a heart, a heart muscle or heart cells by administering a vector encoding a polypeptide or a nucleic acid capable of binding to a phosducin binding site of Gβγ. Said nucleic acid is e.g. an aptamer.

Further, the present invention provides a method of increasing the contractility of a heart, a heart muscle or heart cells by administering a nucleic acid which inhibits expression of a Gβγ component by an anti-sense mechanism.

Further, an active N-terminal truncated phosducin is provided selected from the group of

(a) a polypeptide having the amino acid sequence of SEQ ID NO: 2 or function-conservative variants thereof;

(b) a polypeptide comprising the amino acid sequence of SEQ ID NO: 2, whereby the polypeptide lacks at least 50 N-terminal amino acids of a natural phosducin;

(c) a polypeptide having an amino acid sequence of least 80% identity as compared to the amino acid sequence of SEQ ID NO: 2.

A nucleic acid is provided coding for an active N-terminal truncated phosducin selected from the group of

(a) a nucleic acid having the sequence of SEQ ID NO: 1 or any variant based on the degeneracy of the genetic code;

(b) a nucleic acid the complementary strand of which hybridizes under high stringency conditions with nucleic acids having the nucleic acid sequence of SEQ ID NO:1;

(c) a nucleic acid having a sequence of at least 80 % identity to the sequence of SEQ ID NO: 1.

The present invention further provides screening methods for identifying compounds to be used in the above methods (see below).

The present invention is based on the effect of the intracellular binding of phosducin to Gβγ on the contractility of a heart, a heart muscle or cells of a heart muscle. Moreover, the present invention is based on the recognition of the effect of intracellularly providing phosducin on the sensitivity of a heart, a heart muscle or cells of a heart muscle towards extra-cellular stimuli, such as β-adrenergic receptor agonists like adrenaline. The recognition of the causal connection between an increase of the contractility and sensitivity of a heart, a heart muscle, or cells of a heart muscle, and the binding of phosducin to Gβγ provides a novel approach to the therapy of chronic congestive heart failure. The present invention is particularly surprising in view of the finding that overexpression of full-length phosducin in a mouse disease model of heart failure did not show any positive effect on the development of heart failure (see Reference Example 1).

The examples show that overexpression of an N-terminal truncated phosducin (“nt-del-phosducin”) results in a clear positive inotropic effect in both normal and failing cardiomyocytes after gene transfer ex vivo. Moreover, cardiac function was clearly improved in rabbits with heart failure after in vivo gene transfer of nt-del-phosducin. These results suggest that nt-del-phosducin exerts its positive effects by inhibition of Gβγ-mediated pathways.

To determine the direct functional significance of nt-del-phosducin overexpression on myocardial performance in the absence of tonic sympathoadrenal neural activation and mechanical loading, the contractility of left ventricular myocytes isolated from normal or failing hearts after ex vivo gene transfer were measured (Example 3). A clear increase in isoproterenol-dependent contractility of isolated cardiomyocytes in the presence of nt-del-phosducin was observed. Further, overexpression of nt-del-phosducin enhanced basal contraction and maximal contractility of both, normal and failing cardiomyocytes. Moreover, a clear leftward shift of the concentration-contractility curve occurred (FIG. 5). It was also found that overexpression of nt-del-phosducin increased the contractility and prevented further deterioriation of heart failure after in vivo gene transfer (FIG. 6).

### BRIEF DESCRIPTION OF THE FIGURES

FIG. 1 shows a visualisation of GFP co-expression in myocardial tissue slices. One week after gene transfer of Ad-nt-del-phosducin-GFP, a freeze-cut transverse slice of a rabbit heart under ultraviolet light shows green fluorescence.

FIG. 2. shows a Western blot of cell extracts after gene transfer of Ad-nt-del-phosducin-GFP (lane 2), in comparison to full-length phosducin purified from recombinant E. coli (lane 1) or from a lysate of transfected HEK cells (lane 3), probed with a specific anti-phosducin antibody. Lane 4 shows a lysate from mock-transfected HEK cells as a negative control. The smaller size of nt-del-phosducin (27 kD) can be easily distinguished from full-length phosducin (33 kD).

FIG. 3 shows cAMP formation in adult ventricular cardiomyocytes infected with Ad-GFP, or Ad-nt-del-phosducin-GFP and stimulated with increasing concentrations of isoproterenol. cAMP accumulation is shown as fold increase over baseline. Data represent means±SEM of 5 independent experiments. * p<0.05 vs. GFP.

FIG. 4 shows IP3 accumulation in cardiomyocytes infected with Ad-GFP or Ad-nt-del-phosducin-GFP. Cardiomyocytes were investigated in the absence of agonists (“basal”), after stimulation with 1 μmol/L bradykinin or 10 μmol/l acetylcholine, where indicated. Data represent means±SEM of 5 independent experiments. * p<0.05 vs. nt-del-phosducin.

FIG. 5. Contraction amplitude of cardiomyocytes isolated from healthy hearts (A) or from failing hearts (B). The cardiomyocytes were infected ex vivo with either Ad-GFP or Ad-nt-del-phosducin-GFP. Fractional shortening was determined in response to increasing concentrations of isoproterenol. Data represent means±SEM. At least 25 healthy cells from 8 different hearts were studied in all groups. At least 30 failing cells from 8 different hearts were studied in all groups. * p<0.05 vs GFP; ** p<0.01 vs. GFP. Nonlinear curve fitting using a Hill equation gave the following estimates (EC50 in μmol/L and Emax values, respectively) in healthy cells: GFP 9±1 and 8.7±0.3; nt-del-phosducin: 10±0.1 and 11.2±0.2 (p<0.001 both transgenes vs. GFP); and in failing cells: GFP 21.6±0.3 and 4.7±0.04; nt-del-phosducin 17±0.4 pM and 7.8±0.1 (p<0.001 both transgenes vs. GFP).

FIG. 6 shows hemodynamic function determined by tip catheterization one week after gene transfer of either GFP or nt-del-phosducin. Data represent means±SEM. All measurements were done in 7 animals in triplicates. *p<0.05 vs GFP.

(A) Maximum first derivative of left ventricular pressure (LV dp/dt max) at baseline and in response to isoproterenol.

(B) Maximum left ventricular systolic pressure (mmHg) at baseline and in response to isoproterenol.

FIG. 7. shows echocardiographic determination of fractional shortening. Relative decrease in fractional shortening as assessed by serial echocardiography. The bars show the ratios of FS at the final measurements divided by the measurements before gene transfer in the same animals. All measurements were done in 7 animals in triplicates. *p<0.05 vs GFP.

FIG. 8 Histological alterations in transgenic mouse hearts. Hematoxylin/eosin-stained 5 μm sections of paraffin-embedded left ventricular myocardium from healthy wild-type mice and from a transgenic heart failure model (,,β-TG4”, see Engelhardt et al. (1999) Proc. Natl. Acad. Sci. USA 96, 7059-64). Cross-breeding of this heart failing mouse line with mice which expressed full-length phosducin specifically in their hearts (β1-TG4×Phd-TG) did not result in any histological improvement of the heart failure phenotype (image in the right).

FIG. 9 shows the DNA sequence of full-length human phosducin. Deletion of the 156 N-terminal bases results in SEQ ID No: 1.

FIG. 10 shows the protein sequence of full-length human phosducin in one-letter code. Deletion of the 52 N-terminal amino acids results in SEQ ID No: 2.

### DETAILED DESCRIPTION OF THE INVENTION

Definitions

The following definitions are provided to facilitate understanding of certain terms used frequently herein.

“Phosducin” refers, among others, to a polypeptide comprising the amino acid sequence set forth in SEQ ID NO: 2, or a function-conservative variant thereof including any full-length wild type phosducin molecule. Said phosducin is preferably mammalian, most preferably human.

As used herein an “active N-terminal truncated phosducin” or ,,nt-del-phosducin” refers to a fragment of a full-length wild-type phosducin or a function conservative variant thereof, that contains at least a portion of the C-terminal peptide sequence, but lacks at least a portion of the N-terminal domain of full-length phosducin which can be phosphorylated and thereby inactivated under physiological conditions. Preferably, at least 20, more preferably at least 30 and still more preferably at least 50 N-terminal amino acids are deleted. A specific example for an active N-terminal truncated phosducin is a polypeptide consisting of the amino acid sequence set forth in SEQ ID NO: 2. In addition to the deletion of the N-terminal portion of full-length wild-type phosducin, it is possible to exchange phosphorylatable amino acids for amino acids which are not phosphorylated under physiological conditions in the truncated phosducin. The active N-terminal truncated phosducin is capable of binding to Gβγ, whereby Gβγ-mediated processes are inhibited. As disclosed herein, active N-terminal truncated phosducin can be produced by a number of means including by proteolytic digestion of a phosducin, chemical synthesis and, more preferably, by recombinant DNA techniques. General techniques for constructing nucleic acids that express N-terminal truncated phosducin are conventional molecular biology, microbiology, and recombinant DNA techniques within the state of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); F. M. Ausubel et al. (eds.), Current Protocols in Molecular Biology, John Wiley & Sons, Inc. (1994).

“Gβγ” refers to a protein complex of the P and y subunits of large GTP binding proteins (,,G-proteins”) which are present in the cell membrane and coupled to 7-transmembrane domain receptors. Examples of Gβγ complexes are βγ_{B}, βγ_{T}, β₁γ₃, β₂γ₃, β₂γ, β₁γ₂. Production of such Gβγ complexes is described e.g. in Mulier et al. (1996) J. Biol. Chem. 271, 11781-11786.

“Variant” refers to a polynucleotide or polypeptide that differs from a reference polynucleotide or polypeptide, respectively, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are overall closely similar and, in many regions, identical. A variant and reference polypeptide may differ in amino acid sequence by one or more substitutions, additions, deletions in any combination. A substituted or inserted amino acid residue may or may not be one encoded by the genetic code. A variant of a polynucleotide or polypeptide may be a naturally occurring one such as an allelic variant, or it may be a variant that is not known to occur naturally. Non-naturally occurring variants of polynucleotides and polypeptides may be made by mutagenesis techniques or by direct synthesis.

“Phosducin Activity” or “Biological Activity of Phosducin” refers to the physiologic function of said phosducin on Gβγ including similar activities or improved activities or such activities with decreased undesirable side-effects. Preferably, ,,phosducin activity” refers to the ability of the phosducin variants of the invention to increase the contractility of a heart, a heart muscle or cells of a heart muscle.

“Gβγ Activity” or “Biological Activity of the Gβγ” refers to the physiologic function of said Gβγ on Gβγ-mediated signalling pathways including similar activities or improved activities or such activities with decreased undesirable side-effects. Also included are antigenic and immunogenic activities of said Gβγ.

“Antibodies” as used herein includes polyclonal and monoclonal antibodies, chimeric, single chain, and humanized antibodies, as well as Fab fragments, including the products of a Fab or another immunoglobulin expression library.

“Polynucleotide” or “nucleic acid molecule” generally refers to any polyribonucleotide or polydeoxribonucleotide, which may be unmodified RNA or DNA or modified RNA or DNA. “Polynucleotides” or “nucleic acid molecules” include, single-and double-stranded DNA, DNA that is a mixture of single-and double-stranded regions, single-and double-stranded RNA, and RNA that is mixture of single-and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Polynucleotide” or “nucleic acid molecule”, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5′ to 3′ direction along the nontranscribed strand of DNA (ie., the strand having a sequence homologous to the mRNA). A “recombinant DNA molecule” is a DNA molecule that has undergone a molecular biological manipulation.

According to the invention, nucleic acid molecules can be used for inhibiting expression of a desired Gβγ component by an anti-sense mechanism. Such nucleic acids are preferably RNA-based nucleic acids which may be modified in order to increase their stability in body fluids or cells. Suitable approaches for the preparation of anti-sense RNA are known in the art and are described e.g. in EB-B1 0 223 399.

Aptamers are protein binding nucleic acid molecules which can e.g. be isolated by way of binding affinity to a target protein from large libraries of chemically-modified RNA molecules. For the present invention, aptamers are selected for binding to a phosducin binding site on a Gβγ complex. See Biotechniques (2001) 30, 1094-1096 and references cited therein for methods on obtaining aptamers.

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength (see Sambrook et al., supra). The conditions of temperature and ionic strength determine the “stringency” of the hybridization. For preliminary screening for homologous nucleic acids, low stringency hybridization conditions, corresponding to a Tₘ of 55° C., can be used, e.g., 5×SSC, 0.1% SDS, 0.25% milk and no formamide; or 30% formamide, 5×SSC, 0.5% SDS). Moderate stringency hybridization conditions correspond to a higher Tₘ, e.g., 40% formamide, with 5×or 6×SCC. High stringency hybridization conditions correspond to the highest Tₘ, e.g., 50% formamide, 5×or 6×SCC. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tₘ for hybrids of nucleic acids having those sequences. The relative stability (corresponding to higher Tₘ) of nucleic acid hybridizations decreases in the following order: RNA:RNA, DNA:RNA, DNA:DNA. For hybrids of greater than 100 nucleotides in length, equations for calculating Tₘ have been derived (see Sambrook et al., supra, 9.50-0.51). For hybridization with shorter nucleic acids, i.e., oligonucleotides, the position of mismatches becomes more important, and the length of the oligonucleotide determines its specificity (see Sambrook et al., supra, 11.7-11.8). Preferably a minimum length for a hybridizable nucleic acid is at least about 12 nucleotides; preferably at least about 18 nucleotides; and more preferably the length is at least about 27 nucleotides; and most preferably 36 nucleotides.

In a specific embodiment, the term “standard hybridization conditions” refers to a Tₘ of 55° C., and utilizes conditions as set forth above. In a preferred embodiment, the Tₘ is 60° C.; in a more preferred embodiment, the Tₘ is 65° C.

“Polypeptide” refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids. “Polypeptides” include amino acid sequences modified either by natural processes, such as posttranslational processing, or by chemical modification techniques which are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. The same type of modification may be present in the same or varying degrees at several sites in a given polypeptide. Also, a given polypeptide may contain many types of modifications. Polypeptides may be branched as a result of ubiquitination, and they may be cyclic, with or without branching. Cyclic, branched and branched cyclic polypeptides may result from posttranslation natural processes or may be made by synthetic methods. Modifications include acetylation, acylation, ADP-ribosylation, amidation, covalent attachment of flavin, of a heme moiety, of biotin, fluorescin or another fluorescent dye, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. See, for instance, PROTEINS-STRUCTURE AND MOLECULAR PROPERTIES, 2nd Ed., T. E. Creighton, W. H. Freeman and Company, New York, 1993 and Wold, F., Posttranslational Protein Modifications: Perspectives and Prospects, pgs. 1-12 in POSTTRANSLATIONAL COVALENT MODIFICATION OF PROTEINS, B. C. Johnson, Ed., Academic Press, New York, 1983; Seifter et al., “Analysis for protein modifications and nonprotein cofactors”, Meth Enzymol (1990) 182:626-646 and Rattan et al., “Protein Synthesis: Posttranslational Modifications and Aging”, Ann NY Acad Sci (1992) 663:48-62.

“Identity,” as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those described in (Computational Molecular Biology, Lesk, A. M., ed., Oxford University Press, New York, 1988; Biocomputing: Informatics and Genome Projects, Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Preferred methods to determine identity are designed to give the largest match between the sequences tested. Methods to determine identity and similarity are codified in publicly available computer programs. Preferred computer program methods to determine identity and similarity between two sequences include, but are not limited to, the GCG program package (Devereux, J., et al., Nucleic Acids Research 12(1): 387 (1984)), BLASTP, BLASTN, and FASTA (Atschul, S. F. et al., J. Molec. Biol. 215:403-410 (1990). The BLAST X program is publicly available from NCBI and other sources (BLAST Manual, Altschul, S., et. al., NCBI NLM NIH Bethesda, Md. 20894; Altschul, S., et al., J. Mol. Biol. 215: 403-410 (1990). The well known Smith Waterman algorithm may also be used to determine identity.

Preferred polynucleotide embodiments further include an isolated polynucleotide comprising a polynucleotide having at least 80, 85, 90, 95, 97 or 100% identity to a polynucleotide reference sequence of SEQ ID NO: 1, wherein said reference sequence may be identical to the sequence of SEQ ID NO: 1 or may include up to a certain integer number of nucleotide alterations as compared to the reference sequence, wherein said alterations are selected from the group consisting of at least one nucleotide deletion, substitution, including transition and transversion, or insertion, and wherein said alterations may occur at the 5′ or 3′ terminal positions of the reference nucleotide sequence or anywhere between those terminal positions, interspersed either individually among the nucleotides in the reference sequence or in one or more contiguous groups within the reference sequence.

Preferred polypeptide embodiments further include an isolated polypeptide comprising a polypeptide having at least 80, 85, 90, 95, 97 or 100% identity to the polypeptide reference sequence of SEQ ID NO:2, wherein said reference sequence may be identical to the sequence of SEQ ID NO: 2 or may include up to a certain integer number of amino acid alterations as compared to the reference sequence, wherein said alterations are selected from the group consisting of at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence.

Polypeptides of the Invention

The present invention relates to active N-terminal truncated phosducin polypeptides (nt-del-phosducin). The active N-terminal truncated phosducin polypeptides of the invention include the polypeptide of SEQ ID NO: 2; as well as N-terminal truncated phosducin polypeptides comprising the amino acid sequence of SEQ ID NO: 2; and N-terminal truncated phosducin polypeptides comprising an amino acid sequence which has at least 80% identity to that of SEQ ID NO: 2 over its entire length, and still more preferably at least 90% identity, and even still more preferably at least 95% identity to SEQ ID NO: 2. Furthermore, those with at least 97-99% are highly preferred. The N-terminal truncated phosducin polypeptides exhibit at least Gβγ binding interaction. Preferably, the active N-terminal truncated phosducin polypeptide of the invention is not inactivated by phosphorylation under physiological conditions.

The active N-terminal truncated phosducin polypeptides may be in the form of the “mature” protein or may be a part of a larger protein such as a fusion protein. Said polypeptides may additionally contain secretory or leader sequences, pro-sequences, sequences which aid in purification such as multiple histidine residues, or an additional sequence for stability during recombinant production may also be present.

Fragments of the active N-terminal truncated phosducin polypeptides are also included in the invention. A fragment is a polypeptide having an amino acid sequence that is entirely the same as a part, but not all, of the amino acid sequence of an active N-terminal truncated phosducin polypeptide as mentioned above. As with active N-terminal truncated phosducin polypeptides, fragments may be “free-standing,” or comprised within a larger polypeptide of which they form a part or region, most preferably as a single continuous region. Fragments of active N-terminal truncated phosducin polypeptides must retain biological activity. Biologically active fragments are those that bind to Gβγ and inhibit or dampen Gβγ mediated receptor activity.

Variants of the defined sequence and fragments also form part of the present invention. Preferred variants are those that vary from the reference by conservative amino acid substitutions, i.e., those that substitute a residue with another one of like characteristics. Typical such substitutions are among Ala, Val, Leu and lie; among Ser and Thr; among the acidic residues Asp and Glu; among Asn and Gln; and among the basic residues Lys and Arg; or aromatic residues Phe and Tyr. Particularly preferred are variants in which several, 5-10, 1-5, or 1-2 amino acids are substituted, deleted, or added in any combination.

The active N-terminal truncated phosducin polypeptides of the invention can be prepared in any suitable manner. Such polypeptides include isolated naturally occurring polypeptides, recombinantly produced polypeptides, synthetically produced polypeptides, or polypeptides produced by a combination of these methods.

Nucleic Acids of the Invention

Another aspect of the invention relates to polynucleotides encoding an active N-terminal truncated phosducin. Polynucleotides of the invention include isolated polynucleotides which encode the active N-terminal truncated phosducin polypeptides and fragments thereof, and polynucleotides closely related thereto. More specifically, the polynucleotide of the invention includes a polynucleotide comprising the nucleotide sequence contained in SEQ ID NO: 1 encoding an active N-terminal truncated phosducin polypeptide of SEQ ID NO: 2, and a polynucleotide having the particular sequence of SEQ ID NO: 1. Polynucleotides of the invention further include a polynucleotide comprising a nucleotide sequence that has at least 80% identity over its entire length to a nucleotide sequence encoding the active N-terminal truncated phosducin polypeptide of SEQ ID NO: 2, and a polynucleotide comprising a nucleotide sequence that is at least 80% identical to that of SEQ ID NO: 1 over its entire length. In this regard, polynucleotides which are at least 90% identical to SEQ ID NO: 1 are particularly preferred, and those with at least 95% identity are especially preferred. Furthermore, those with at least 97% identity are highly preferred and those with at least 98-99% identity are most highly preferred, with at least 99% being the most preferred. Also included under polynucleotides of the invention is a nucleotide sequence the complementary strand of which hybridizes to a nucleotide sequence contained in SEQ ID NO: 1 under conditions useable for amplification or for use as a probe or marker. The invention also provides polynucleotides which are complementary to such polynucleotides.

A polynucleotide of the present invention encoding active N-terminal truncated phosducin may be obtained using standard cloning and screening, from a cDNA library derived from mRNA using the expressed sequence tag (EST) analysis (Adams, M. D., et al. Science (1991) 252:1651-1656; Adams, M. D. et al., Nature, (1992) 355:632-634; Adams, M. D., et al., Nature (1995) 377 Supp:3-174). Polynucleotides of the invention can also be obtained from natural sources such as genomic DNA libraries or can be synthesized using well known and commercially available techniques.

The nucleotide sequence encoding the active N-terminal truncated phosducin of SEQ ID NO: 2 may be identical to a sequence contained in SEQ ID NO: 1 or it may be a sequence which, as a result of the redundancy (degeneracy) of the genetic code, also encodes the polypeptide of SEQ ID NO: 2.

When the polynucleotides of the invention are used for the recombinant production of active N-terminal truncated phosducin, the polynucleotide may include the coding sequence for the mature polypeptide or a fragment thereof; the coding sequence for the mature polypeptide or fragment in reading frame with other coding sequences, such as those encoding a leader or secretory sequence, a pre-, or pro- or prepro-peptide sequence, or other fusion peptide portions. For example, a marker sequence which facilitates purification of the fused polypeptide can be encoded. In certain preferred embodiments of this aspect of the invention, the marker sequence is a hexahistidine peptide, as provided in the pQE vector (Qiagen, Inc.) and described in Gentz et al., Proc Natl Acad Sci USA (1989) 86:821-824, or is an HA tag. The polynucleotide may also contain noncoding 5′ and 3′ sequences, such as transcribed, non-translated sequences, splicing and polyadenylation signals, ribosome binding sites and sequences that stabilize mRNA.

Further preferred embodiments are polynucleotides encoding active N-terminal truncated phosducin variants comprising the amino acid sequence of SEQ ID NO: 2 in which several, 5-10, 1-5, 1-3, 1-2 or 1 amino acid residues are substituted, deleted or added, in any combination.

The present invention further relates to polynucleotides that hybridize to the herein above-described sequences. In this regard, the present invention especially relates to polynucleotides which hybridize under stringent conditions to the herein above-described polynucleotides. As herein used, the term “stringent conditions” means hybridization will occur only if there is at least 80%, and preferably at least 90%, and more preferably at least 95%, yet even more preferably 97-99% identity between the sequences.

Polynucleotides of the invention, which are identical or sufficiently identical to a nucleotide sequence contained in SEQ ID NO: 1 or a fragment thereof, may be used as hybridization probes for cDNA and genomic DNA, to isolate full-length cDNAs and genomic clones encoding active N-terminal truncated phosducin and to isolate cDNA and genomic clones of other genes (including genes encoding homologs and orthologs from species other than human) that have a high sequence similarity to the active N-terminal truncated phosducin gene. Such hybridization techniques are known to those of skill in the art. Typically these nucleotide sequences are 80% identical, preferably 90% identical, more preferably 95% identical to that of the referent. The probes generally will comprise at least 15 nucleotides. Preferably, such probes will have at least 30 nucleotides and may have at least 50 nucleotides. Particularly preferred probes will range between 30 and 50 nucleotides.

In one embodiment, obtaining a polynucleotide encoding active N-terminal truncated phosducin, including homologs and orthologs from species other than human, comprises the steps of screening an appropriate library under stingent hybridization conditions with a labeled probe having the SEQ ID NO: 1 or a fragment thereof; and isolating full-length cDNA and genomic clones containing said polynucleotide sequence. Thus in another aspect, active N-terminal truncated phosducin polynucleotides of the present invention further include a nucleotide sequence comprising a nucleotide sequence that hybridizes under stringent condition to a nucleotide sequence having SEQ ID NO: 1 or a fragment thereof. Also included with active N-terminal truncated phosducin polypeptides is a polypeptide comprising an amino acid sequence encoded by a nucleotide sequence obtained by the above hybridization condition. Such hybridization techniques are well known to those of skill in the art. Stringent hybridization conditions are as defined above or, alternatively, conditions under overnight incubation at 42° C. in a solution comprising: 50% formamide, 5×SSC (150 mM NaCl, 15 mM trisodium citrate), 50 mM sodium phosphate (pH7.6), 5×Denhardt's solution, 10% dextran sulfate, and 20 microgram/ml denatured, sheared salmon sperm DNA, followed by washing the filters in 0.1×SSC at about 65° C.

The polynucleotides and polypeptides of the present invention may be employed as research reagents and materials for discovery of treatments and diagnostics to animal and human disease.

Vectors, Host Cells, Expression

The present invention also relates to vectors which comprise a polynucleotide or polynucleotides of the present invention, and host cells which are genetically engineered with vectors of the invention and to the production of polypeptides of the invention by recombinant techniques. Cell-free translation systems can also be employed to produce such proteins using RNAs derived from the DNA constructs of the present invention.

For recombinant production, host cells can be genetically engineered to incorporate expression systems or portions thereof for polynucleotides of the present invention. Introduction of polynucleotides into host cells can be effected by methods described in many standard laboratory manuals, such as Davis et al., BASIC METHODS IN MOLECULAR BIOLOGY (1986) and Sambrook et al., MOLECULAR CLONING: A LABORATORY MANUAL, 2nd Ed., Cold Spring Harbor Laboratory Press, Cold Spring Harbor, N.Y. (1989) such as calcium phosphate transfection, DEAE-dextran mediated transfection, microinjection, cationic lipid-mediated transfection, electroporation, transduction, scrape loading, ballistic introduction or infection.

Representative examples of appropriate hosts include bacterial cells, such as streptococci, staphylococci, E. coli, Streptomyces and Bacillus subtilis cells; fungal cells, such as yeast cells and Aspergillus cells; insect cells such as Drosophila S2 and Spodoptera Sf9 cells; animal cells such as CHO, COS, HeLa, C127, 3T3, BHK, HEK 293 and Bowes melanoma cells; and plant cells.

A great variety of expression systems can be used. Such systems include, among others, chromosomal, episomal and virus-derived systems, vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. The expression systems may contain control regions that regulate as well as engender expression. Generally, any system or vector suitable to maintain, propagate or express polynucleotides to produce a polypeptide in a host may be used.

The appropriate nucleotide sequence may be inserted into an expression system by any of a variety of well-known and routine techniques, such as, for example, those set forth in Sambrook et al., MOLECULAR CLONING, A LABORATORY MANUAL.

For secretion of the translated protein into the lumen of the endoplasmic reticulum, into the periplasmic space or into the extracellular environment, appropriate secretion signals may be incorporated into the desired polypeptide. These signals may be endogenous to the polypeptide or they may be heterologous signals.

If the active N-terminal truncated phosducin is to be expressed for use in screening assays, generally, it is preferred that the polypeptide be produced at the surface of the cell. In this event, the cells may be harvested prior to use in the screening assay. If active N-terminal truncated phosducin polypeptide is secreted into the medium, the medium can be recovered in order to recover and purify the polypeptide; if produced intracellularly, the cells must be lysed to recover the polypeptide.

Active N-terminal truncated phosducin polypeptides can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, fast protein liquid chromatography (FPLC) is employed for purification. Well known techniques for refolding proteins may be employed to regenerate an active conformation when the polypeptide is denatured during isolation and or purification.

Screening Assays

Binding to a phosducin binding site of Gβγ in myocardial cells is responsible for an increase of contractility and sensitivity of a heart, a heart muscle or cells of a heart muscle . Accordingly, it is desirous to find compounds and drugs which can inhibit the function of Gβγ in myocardial cells. In general, antagonists of Gβγ signalling pathways identified according to the invention are employed for therapeutic purposes for the treatment of congestive heart failure.

The screening for compounds and drugs which can inhibit the function of Gβγ in myocardial cells by binding to a binding site of phosducin may be conducted by rational drug design based on the protein structure of Gβγ. The crystal structure at 2.4 Å resolution of the complex of Gβγ and Phosducin has been published (Gaudet et al., Cell, 87, 577-588, (1996)) and the coordinates of the phosducin/Gβγ structure are available from the Protein Data Base (entries 1A0R, 1B9X, 1B9Y, and 2TRC). Moreover, further crystal structures of Gβγ or the interaction of Gβγ and phosducin or an active N-terminal truncated phosducin are available according to general methods known in the art. Based on the 3-D structure of Gβγ, a potential drug or agent can be examined through the use of computer modeling using a standard docking program such as GRAM, DOCK, or AUTODOCK (Goodsell et al. (1990) Proteins: Structure, Function and Genetics, 8, 195-201; Kuntz et al. (1982) J. Mol. Biol. 161, 269-288). This procedure can include computer fitting of potential agents to the Gβγ. Computer methods can also be employed to estimate the attraction, repulsion, and steric hindrance of the agent to a phosducin binding site of Gβγ. Generally, the tighter the fit (e.g., the lower the steric hindrance, and/or the greater the attractive force) the more potent the potential drug will be since these properties are consistent with a tighter binding constant. Furthermore, the higher the specificity of a potential drug, the more likely it is that the drug will not interfere with related proteins, thereby minimizing potential side-effects due to unwanted interactions with other proteins.

Compounds and drugs may bind to any phosducin binding site of Gβγ. Preferably, such compounds bind to the binding site of an nt-del-phosducin, most preferably to a binding site of the polypeptide of SEQ ID NO: 2. These binding sites can be obtained from the work of Gaudet et al. (supra).

Initially, a potential drug could be obtained by screening a peptide library produced based on N-terminal truncated phosducin or a chemical library. An agent selected in this manner could then be systematically modified by computer modeling programs until one or more promising potential drugs are identified. Examples for this strategy are known from Lam et al., Science 263:380-384 (1994); Wlodawer et al., Ann. Rev. Biochem. 62:543-585 (1993); Appelt, Perspectives in Drug Discovery and Design 1:23-48 (1993). Such computer modeling allows the selection of a number of rational chemical modifications, as opposed to the countless number of essentially random chemical modifications that could be made. Thus, by using three-dimensional structural analysis and computer modeling, a large number of these compounds can be rapidly screened computationally, and a few likely candidates can be determined without laborious synthesis.

Once a potential drug is identified it can be either selected from a commercially available library of chemicals as are commercially available from most large chemical companies. The de novo synthesis of one or even a relatively small group of specific compounds is reasonable in the art of drug design and cannot be considered as an undue burden on the way to an active agent.

The potential drug can then be tested e.g. in a competitive binding assay (including in high throughput binding assays) for its ability to bind to Gβγ in the presence of an active N-terminal truncated phosducin of the invention. Alternatively the potential drug can be tested for: (1) its ability to increase the contractility of muscle cells in a screening assay according to the invention; or (2) its ability to inhibit Gβγ-mediated processes. When a suitable potential drug is identified, a second structural analysis can optionally be performed on the binding complex formed between the Gβγ and the potential drug.

In a specific embodiment of the screening assay of the invention, the screening comprises the following steps:

(a) incubating a mixture comprising a predetermined concentration of Gβγ and a predetermined concentration of phosducin, an N-terminal truncated phosducin, or a function conservative variant under conditions which allow for binding of the phosducin, the N-terminal truncated phosducin, or the function conservative variant to Gβγ,

(b) incubating a mixture according to (a) under conditions according to (a) in the presence of a predetermined concentration of a test compound potentially capable of binding to Gβγ, and

(c) selecting a test compound providing a higher concentration of phosducin, of said N-terminal truncated phosducin or of said variant not bound to Gβγ in the mixture of step (b) than the mixture of step (a).

In general, such screening procedures involve providing appropriate mixtures containing Gβγ. Such mixtures include sub-cellular mixtures or mixtures prepared based on a purified phosducin variant and components of G-protein-mediated signalling pathways.

According to a specific embodiment, a compound able to bind to the phosducin binding site of Gβγ can be specifically designed through NMR based methodology according to Shuker et al., Science 274:1531-1534 (1996). In one such embodiment, a specific compound or a library of low molecular weight compounds is screened to identify a binding partner for Gβγ. Any compound or any chemical library can be used. The assay starts with contacting a mixture containing an active phosducin with a ¹⁵N-labeled Gβγ. Binding of the phosducin to Gβγ can be determined by monitoring the ¹⁵N- or ¹H-amide chemical shift changes in two dimensional ¹⁵N-heteronuclear single-quantum correlation (¹⁵N-HSQC) spectra. A further mixture containing one or more test compounds is then contacted with a ¹⁵N-labeled Gβγ and binding of the test compound to Gβγ can be determined as above. Since such NMR spectra can be rapidly obtained, it is feasible to screen a large number of compounds. A compound is identified as a potential ligand if it binds to Gβγ according to phosducin. In a further step, the test compound is tested as to whether it is able to provide a higher concentration of free unbound phosducin, N-terminal truncated phosducin or variant in the mixture as compared to the mixture not containing the test compound. The structure of the test compound can then be used as a model structure, and analogs to the compound can be obtained (e.g, from the vast chemical libraries commercially available, or alternatively through de novo synthesis). The analogs are then again screened for their ability to bind to Gβγ and to provide a higher concentration of free unbound phosducin, N-terminal truncated phosducin or variant in the mixture as compared to the mixture not containing the test compound. An analog of the initial test compound is chosen as an improved test compound if it binds to Gβγ with a higher binding affinity than the potential ligand. In a preferred embodiment of this type the analogs are screened by ¹⁵N-HSQC-spectroscopy upon addition of the analog to ¹⁵ N-labeled Gβγ as described above.

Furthermore, compounds may be screened for binding to two nearby phosducin binding sites on an Gβγ. In this case, a compound is first identified that binds a first site of Gβγ, but does not bind to a second nearby site. Binding to the second site can be determined by monitoring changes in a different set of amide chemical shifts in either the original screen or a second screen conducted in the presence of a test compound (or potential ligand) for the first site. From an analysis of the chemical shift changes, the approximate location of a potential ligand for the second site is identified. Optimization of the second ligand for binding to the site is then carried out by screening structurally related compounds (e.g. analogs as described above). When ligands for the first site and the second site are identified, their location and orientation in the ternary complex can be determined experimentally either by NMR spectroscopy or X-ray crystallography. On the basis of this structural information, a linked compound is synthesized in which the ligand for the first site and the ligand for the second site are linked. In a preferred embodiment of this type the two ligands are covalently linked. This linked compound is tested to determine if it has a higher binding affinity for Gβγ than either of the two individual ligands and higher binding affinity than active phosducin. A linked compound is selected if it has a higher binding affinity for Gβγ than either of the two ligands or active phosducin. In a preferred embodiment of this type, the test compounds are screened by ¹⁵ N-HSQC-spectroscopy upon addition of the test compound to ¹⁵N-labeled Gβγ as described above.

Any Gβγ protein known from the prior art may be used in such an NMR drug screening procedure. In addition, a larger linked compound can be constructed in an analogous manner, e.g., by linking three ligands which bind to three nearby sites on Gβγ to form a multi-linked compound that has an even higher affinity for Gβγ than a linked compound.

In another assay, Gβγ is placed on or coated onto a solid support. Methods for placing the peptides or proteins on a solid support are well known in the art and include means as linking biotin to the protein and linking avidin to the solid support. An active phosducin which may be labelled is added under conditions allowing for binding of the active phosducin to Gβγ, and allowed to equilibrate. Subsequently, a test compound is allowed to equilibrate with the Gβγ/phosducin complex to test for competitive binding.

The active phosducin or the test compound may be labeled. For example, in one embodiment radiolabeled compounds are used to measure the binding of the compound. In another embodiment, the compounds have fluorescent markers. In yet another embodiment, a Biocore chip (Pharmacia) coated with Gβγ is used and the change in surface conductivity can be measured. In a further embodiment, radiolabeled active phosducin is used to measure the binding of the compound. In another embodiment the active phosducin carries a fluorescent marker.

The effect of a test compound on Gβγ may also be assayed in a living cell that contains Gβγ. Specifically, the present invention provides a method of identifying a compound which increases the contractility of muscle cells, comprising the following steps:

(a) measuring the contractility of isolated muscle cells after stimulation, preferably with a β-adrenergic receptor agonist,

(b) measuring the contractility of isolated muscle cells according to (a), whereby said muscle cells are further exposed to a test compound potentially increasing the contractility of the muscle cells, and

(c) selecting a test compound which causes a higher contractility in step (b) than in step (a).

In particular, a polynucleotide encoding a phosducin of the present invention may be employed to transfect heart muscle cells to express an active N-terminal truncated phosducin polypeptide. The cells are then contacted with a test compound to observe binding, stimulation or inhibition of a functional response.

The prospective drug is tested under conditions in which Gβγ0 signalling is activated, e.g. by providing a β-adrenergic receptor agonist (e.g. adrenaline, noradrenaline). A test compounds which causes a higher contractility in step (b) than in step (a) above is selected. Any muscle cell may be used, preferably a heart muscle cell.

Other screening techniques include a method for identifying a compound which inhibits Gβγ-mediated processes, comprising the following steps:

(i) incubating a mixture comprising Gβγ and a downstream component of a Gβγ-mediated process in pre-defined concentrations, whereby said component is controlled by a Gβγ mediated process in the mixture,

(ii) incubating, under conditions as in (i), a mixture comprising Gβγ, said downstream component of a Gβγ-mediated process and a test compound which potentially inhibits Gβγ-mediated processes, and

(iii) selecting a test compound which inhibits Gβγ in said Gβγ-mediated process.

Moreover, a method of identifying a compound which inhibits Gβγ-mediated processes in cells, comprising the following steps:

(i) incubating cells with an agonist of a G-protein-coupled receptor and measuring a signal due to the amount or activity of a component of a Gβγ-mediated process,

(ii) incubating cells, under conditions as in (i) with said agonist and a test compound which potentially inhibits Gβγ-mediated processes and measuring said signal due to the amount or activity of said component of said Gβγ-mediated process, and

(iii) selecting a test compound which results in a lower amount or activity of said component in step (ii) than in step (i).

The amount (and/or activity) of a reporter produced in the absence and presence of a test compound is determined and compared. A preferred reporter is inositol 1,4,5-triphosphate (IP3) which can be quantified using a commercial kit. Test compounds which reduce the amount (and/or activity) of reporter produced are candidate antagonists of the N-terminal interaction.

In these techniques, compounds may be contacted with mixtures or cells, whereby a second messenger response, e.g. IP3, cAMP or Ca²⁺, is then measured to determine whether the potential compound activates or inhibits Gβγ.

The present invention also provides antagonists obtainable from the above described screening methods.

Examples of potential compounds (antagonists) capable of binding to a phosducin binding site of Gβγ include peptidomimetics, synthetic organic molecules, natural products, antibodies, or nucleic acids (e.g. aptamers, intramers). Examples of small molecule antagonists include small peptides, peptide-like molecules or non-peptide molecules.

For all of the drug screening assays described herein further refinements to the structure of the drug will generally be necessary and can be made by the successive iterations of any and/or all of the steps provided by the particular drug screening assay, including further structural analysis by NMR, for example.

The present invention also relates to the use of active N-terminal truncated phosducin polypeptides as reagents in screening assays of identifying a compound capable of binding to Gβγ . In particular, the N-terminal truncated phosducin polypeptide of the present invention may be employed in a process for screening for compounds which bind to and inhibit Gβγ(called antagonists). Thus, N-terminal truncated phosducin polypeptides of the invention may be used to assess the binding of small molecules and ligands in, for example, cells, cell-free preparations, chemical libraries, and natural product mixtures containing Gβγ. These small molecules and ligands may be natural molecules or may be structural or functional mimetics. See Coligan, et al., Current Protocols in Immunology (2):Chapter 5 (1991).

The present invention also provides methods of rational drug design which may be used for de novo identification of Gβγ-binding drugs (antagonists) or for further refinement of existing antagonists as mentioned above.

Specifically, the present invention provides a method of identifying a compound which increases the contractility of muscle cells, comprising the following steps:

(i) obtaining a set of atomic coordinates defining the three-dimensional structure of the binding site of phosducin to a Gβγ protein complex

(ii) selecting a test compound by performing rational drug design with the atomic coordinates obtained in step (i), wherein said selecting is performed in conjunction with computer modeling;

(iii) contacting the potential agent with a muscle cell; and

(iv) measuring the contractility under predetermined conditions under which the muscle cell has a predetermined contractility;

wherein a test compound is identified as a compound that increases contractility when there is a higher contractility in the presence of the test compound relative to in its absence.

Moreover, the present invention provides a method of identifying a compound for use as an inhibitor of Gβγ-mediated processes comprising:

(i) obtaining a set of atomic coordinates defining the three-dimensional structure of the binding site of phosducin to a Gβγ protein complex;

(ii) selecting a test compound by performing rational drug design with the atomic coordinates obtained in step (i), wherein said selecting is performed in conjunction with computer modeling;

(iii) contacting the test compound with a Gβγ in a mixture allowing for Gβγ-mediated processes; and

(iv) measuring a Gβγ-mediated process;

wherein a test compound is identified as a compound that inhibits Gβγ-mediated processes when there is a decrease in the activity of the Gβγ-mediated process in the presence of the test compound relative to in its absence.

The present invention also relates to an assay kit for identifying a compound capable of binding to Gβγ, comprising active N-terminal truncated phosducin polynucleotide. Specifically, the screening kit for identifying compounds capable of binding to Gβγ comprises:

(b) an active N-terminal truncated phosducin polypeptide, preferably that of SEQ ID NO: 2; which is preferably labeled; and/or

(c) a Gβγ molecule.

Antibodies Against Gβγ

Gβγ can also be used as immunogen to produce antibodies immunospecific for Gβγ. The term “immunospecific” means that the antibodies have substantially greater affinity for Gβγ than for other related polypeptides in the prior art.

Antibodies generated against Gβγ can be obtained by administering the polypeptides or epitope-bearing fragments, analogs or cells to an animal, preferably a nonhuman, using routine protocols. For preparation of monoclonal antibodies, any technique which provides antibodies produced by continuous cell line cultures can be used. Examples include the hybridoma technique (Kohler, G. and Milstein, C., Nature (1975) 256:495-497), the trioma technique, the human B-cell hybridoma technique (Kozbor et al., Immunology Today (1983) 4:72) and the EBV-hybridoma technique (Cole et al., MONOCLONAL ANTIBODIES AND CANCER THERAPY, pp. 77-96, Alan R. Liss, Inc., 1985). Techniques for the production of single chain antibodies (U.S. Pat. No. 4,946,778) can also be adapted to produce single chain antibodies to polypeptides of this invention. Also, transgenic mice, or other organisms including other mammals, may be used to express humanized antibodies.

The above-described antibodies may be employed to isolate or to identify clones expressing the polypeptide or to purify the polypeptides by affinity chromatography.

Antibodies against Gβγ may further be employed to treat or prevent congestive heart failure.

Therapeutic Methods

This invention provides methods for the treatment of congestive heart failure by increasing the contractility of a heart, a heart muscle or cells of a heart muscle by administering an agent capable of binding to a phosducin binding site of Gβγ.

If the activity of Gβγ is too high or if the contractility of the heart is unsufficient, several approaches are available. One approach comprises administering to a subject an inhibitor compound (antagonist) as hereinabove described along with a pharmaceutically acceptable carrier in an amount effective to inhibit activation of the G protein by binding to the Gβγ. Nucleic acids for anti-sense technology or aptamers may either be administered directly or they may be produced in vivo using gene therapy.

Gene therapy may further be employed to effect the endogenous production of active N-terminal truncated phosducin by the relevant cells in the subject. For example, a polynucleotide of the invention may be engineered for expression of an active N-terminal truncated phosducin in a replication defective viral vector. A viral expression construct may be isolated and introduced into a packaging cell transduced e.g. with an adenoviral plasmid vector containing DNA encoding a polypeptide or nucleic acid gene product according to the invention. With a helper virus, the packaging cell can produce infectious viral particles containing the gene of interest. These producer cells may be administered to a subject for engineering cells in vivo and expression of the polypeptide in vivo. For overview of gene therapy, see Chapter 20, Gene Therapy and other Molecular Genetic-based Therapeutic Approaches, (and references cited therein) in Human Molecular Genetics, T Strachan and A P Read, BIOS Scientific Publishers Ltd (1996). Another approach is to administer a therapeutic amount of an active N-terminal truncated phosducin polypeptide in combination with a suitable pharmaceutical carrier.

Formulation and Administration

Peptides, such as an active N-terminal truncated phosducin, antagonist peptides, small molecules or nucleic acid drugs, may be formulated in combination with a suitable pharmaceutical carrier. Such formulations comprise a therapeutically effective amount of the polypeptide or compound, and a pharmaceutically acceptable carrier or excipient. Such carriers include but are not limited to, saline, buffered saline, dextrose, water, glycerol, ethanol, liposomes and suitable combinations thereof. The formulation should suit the mode of administration, and is well within the skill of the art. The invention further relates to pharmaceutical packs and kits comprising one or more containers filled with one or more of the ingredients of the aforementioned compositions of the invention.

Polypeptides and other compounds of the present invention may be employed alone or in conjunction with other compounds, such as therapeutic compounds.

Preferred forms of systemic administration of the pharmaceutical compositions include injection, typically by intravenous injection. Other injection routes, such as subcutaneous, intramuscular, or intraperitoneal, can be used. Alternative means for systemic administration include transmucosal and transdermal administration using penetrants such as bile salts or fusidic acids or other detergents. In addition, if properly formulated in enteric or encapsulated formulations, oral administration may also be possible. Administration of these compounds may also be topical and/or localized, in the form of salves, pastes, gels and the like.

The dosage range required depends on the choice of peptide, the route of administration, the nature of the formulation, the nature of the subject's condition, and the judgment of the practitioner. Suitable dosages, however, are in the range of 0.1-100 mg/kg of subject. Wide variations in the needed dosage, however, are to be expected in view of the variety of compounds available and the differing efficiencies of various routes of administration. For example, oral administration would be expected to require higher dosages than administration by intravenous injection. Variations in these dosage levels can be adjusted using standard empirical routines for optimization, as is well understood in the art.

Polypeptides used in treatment can also be generated endogenously in the subject, in treatment modalities often referred to as “gene therapy” as described above. Thus, for example, cells from a subject may be engineered with a polynucleotide, such as a DNA or RNA to encode a polypeptide ex vivo, and for example, by the use of a virus-base vector. The cells are then introduced into the subject.

Alternatively, a replication-deficient viral vector containing a polynucleotide encoding an N-terminal truncated phosducin may be administered to a patient. Such a viral vector may be an adenovial vector, most preferably a gutless adenoviral vector (for an overview on gutless vectors see Kochanek (1999), Human Gene Therapy 10, 2451-2459).

### EXAMPLES

The invention is now further illustrated based on the following examples. All data are expressed as means±standard error of the means (SEM). For statistical analysis, analysis of variance (ANOVA) for repeated measurements followed by Scheffe's testing, or, where appropriate, Student's t-test with two-tailed distribution, was used. For all analyses, a value of P<0.05 was considered to be statistically significant.

Expression and purification of various G protein βγ-subunits was performed as described by Muller et al. (1996) J. Biol. Chem. 271, 11781-11786.

In the following examples ,,nt-del-phosducin” refers to the specific phosducin variant of SEQ ID No: 2.

### Reference Example 1

### Cloning of a Transgenic Mouse Over Expressing Full-Length Phosducin

The sequence of full-length phosducin was cloned into a plasmid as described by Engelhardt et al. (1999) Proc. Natl. Acad. Sci. USA 96, 7059-64. The purified linear DNA (1 μg/μl) was injected into fertilized oocytes from superovulated FVB/N mice according to standard procedures. The injected oocytes were transferred to the oviducts of pseudopregnant CD-1 mice. All mice were kept in a specific pathogen-free facility. Generation and investigation of these mice was approved by the responsible government authorities. The F0 generation was screened for integration of the transgene by PCR using specific primers.

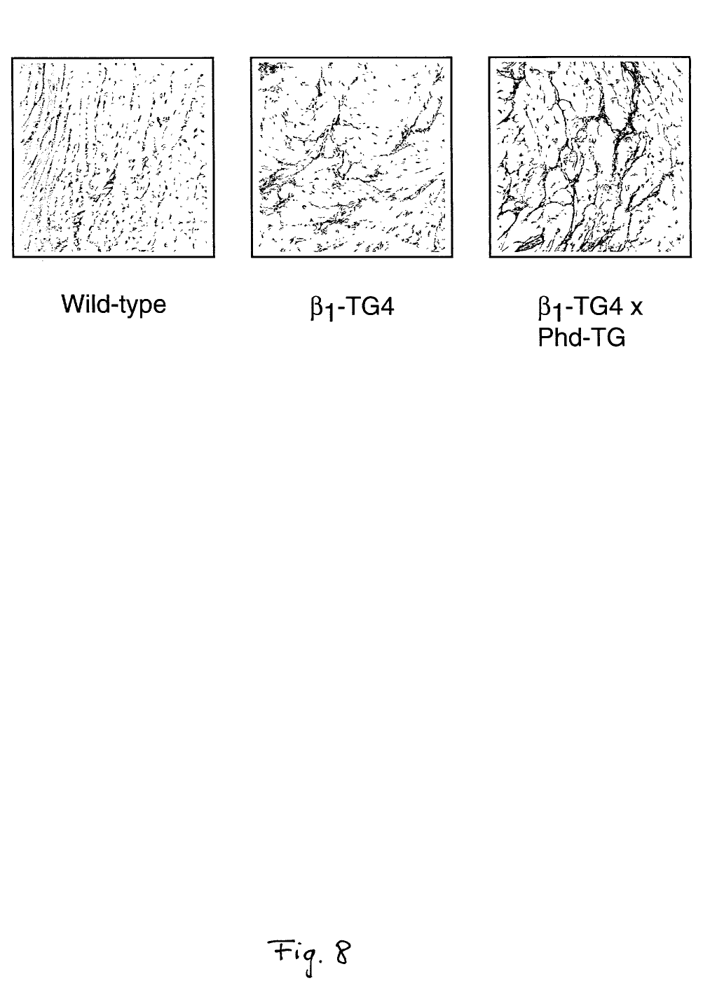

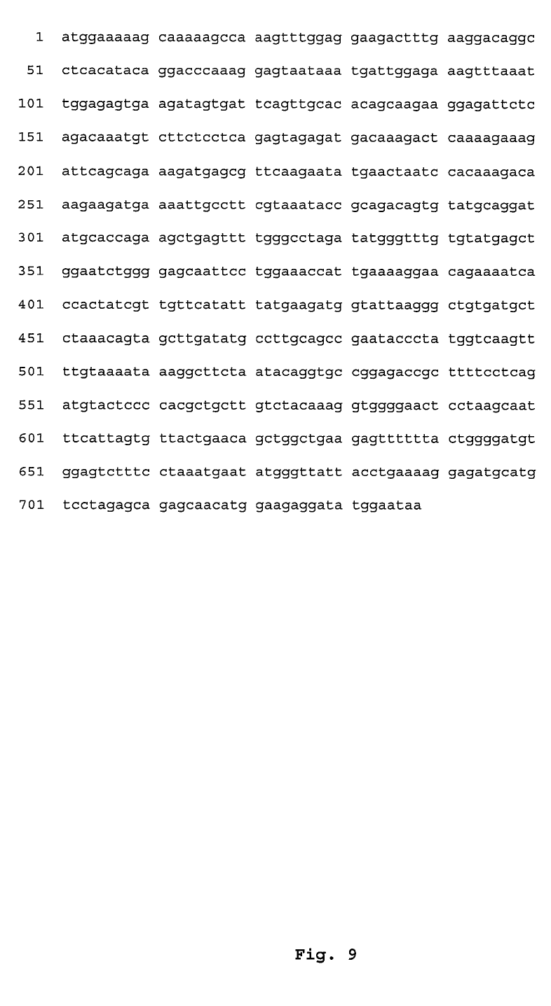

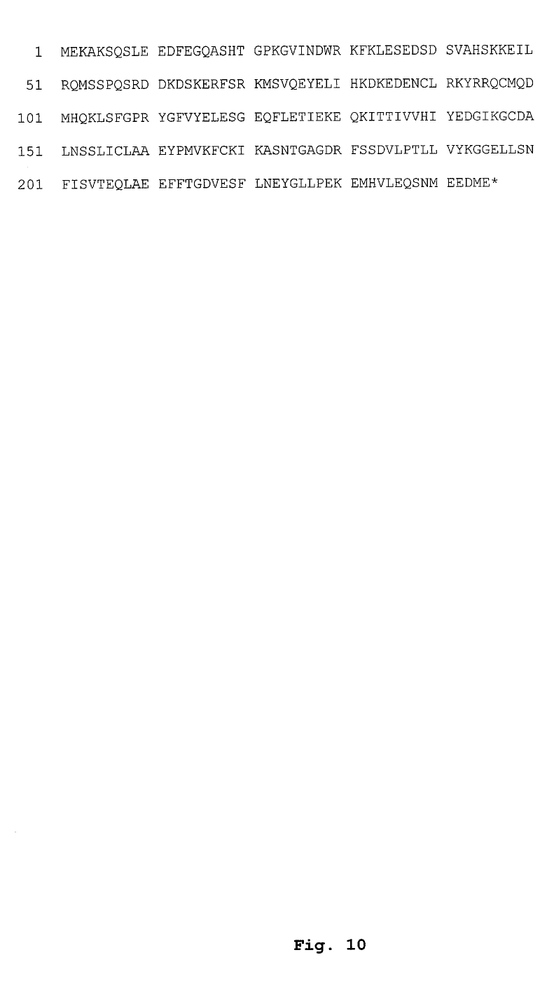

The transgenic mouse was tested with regard to the effect of overexpression of wild type full-length phosducin on the development of heart failure in a murine disease model. It was found that no observable differences in the degree of heart failure (determined histologically as myocyte hypertrophy and fibrosis of heart sections) exist between heart-failing mice and heart-failing mice cross-bred with mice overexpressing full-length phsoducin prepared as described above (FIG. 8 and legend thereto).

### Example 1

### Construction and Purification of Recombinant Adenovirus

Recombinant (E1/E3-Deficient) flag-tagged adenovirus for nt-del-phosducin (Ad-nt-del-phosducin-GFP) was generated, expressing the transgene and green fluorescence protein (GFP) under the control of two independent CMV promoters in a bi-cistronic system (He et al. (1998) Proc Natl Acad Sci USA 95, 2509-14). As a control, Ad-GFP without further transgenes was used. Large virus stocks were prepared as described previously (Laugwitz et al. (1999) Circulation 99, 925-933). Adenoviral titers were determined using plaque titration and GFP expression titration in non E1-expressing cells.

### Example 2

### Preparation and Culture of Adult Ventricular Cardiomyocytes and Adenovirus Infection

Single calcium-tolerant ventricular myocytes were isolated from White New Zealand rabbits. Cardiomyocytes from healthy or failing rabbit hearts were isolated according to the same protocol (Laugwitz et al. (2001) Circ Res. 88, 688-95). Briefly, the hearts were perfused and digested with collagenase. The isolated cardiomyocytes were then resuspended and cultured in modified M199 on laminin-precoated dishes (5-10 μg/cm²) at a density of 1.5×10⁵ cells per cm² (at 5% CO₂ and 37° C.). The cells were infected with adenovirus (multiplicity of infection (moi) 1 pfu/cell) 5 hours after plating. 50-60% of the infected cardiomyocytes express the transgene at this titer. Cardiomyocytes were harvested 48 hours after adenoviral infection. The cells were homogenized and cytosolic extracts were then used for western bloffing by using a polyclonal rabbit antibody raised against phosducin. Goat anti-rabbit second antibodies by Dianova, Germany, were used as second antibodies.

### Example 3

### Functional Consequences of nt-del-phosducin Binding to Gβγ in Isolated Cardiomyocytes

Single cell contraction after adenovirus delivery: Contractility of infected cardiomyocytes was measured by an electro-optical monitoring system connected to online digitalized assessment of amplitude and velocity of shortening and of relaxation as described before (Laugwitz et al. (2001) Circ Res. 88, 688-95). After the contraction amplitude reached stability, increasing concentrations of isoproterenol were applied.

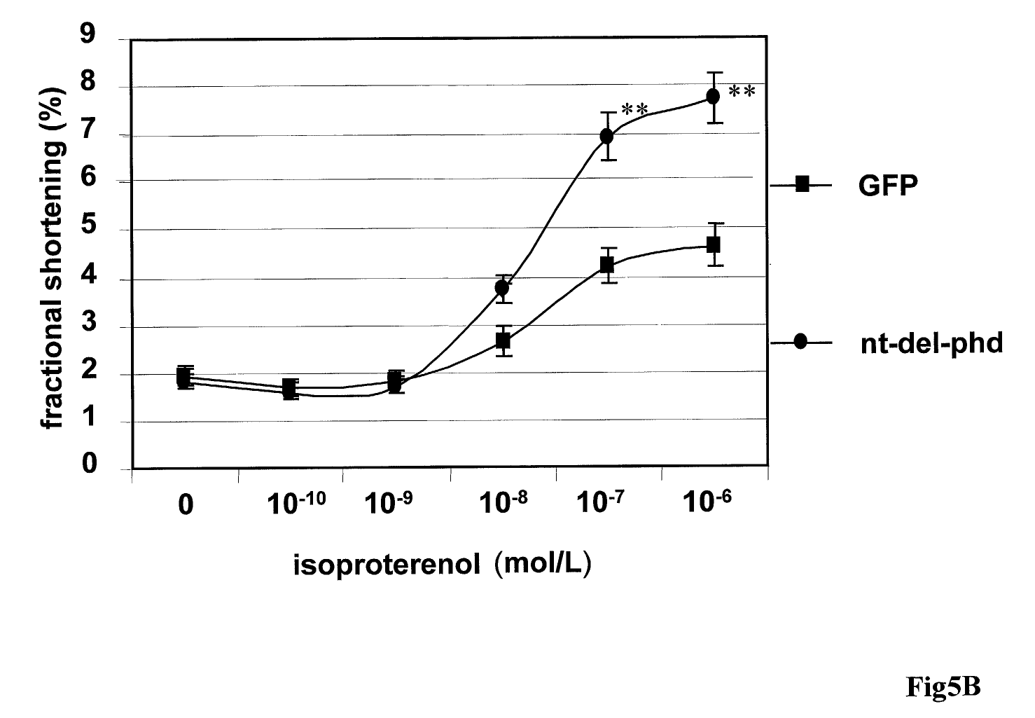

The effects of nt-del-phosducin on cardiomyocyte contractility is demonstrated by the measurement of fractional shortening and velocity of shortening in single, isolated cardiomyocytes from both failing and normal hearts after ex vivo gene transfer. Compared to Ad-GFP-infected control cells, basal and maximal contractility in response to isoproterenol were markedly increased in nt-del-phosducin-expressing cardiomyocytes (FIG. 5A). Overexpression of nt-del-phosducin also enhanced maximal contraction amplitude of failing cardiomyocytes in response to isoproterenol (FIG. 5B). Very similar results were obtained for shortening velocity. The concentration-response curves of nt-del-phosducin-expressing normal and failing cells were significantly shifted to the left. In all batches of virus-infected cells, also GFP-negative cardiomyocytes (which did not express the transgenes) were tested for contractility. These cells did not show any difference compared to non-infected cells, thus demonstrating the comparability of preparation quality.

Intracellular cAMP formation in cardiomyocytes: The effects of nt-del-phosducin expression on G protein-mediated signalling are shown based on the measurement of cAMP accumulation in isolated cardiomyocytes after ex vivo gene transfer. Cardiomyocytes were investigated 48 hours after adenoviral infection. The cells were harvested and stimulated with increasing concentrations of isoproterenol for 20 minutes. The reaction was stopped by adding 100 μl of a 20 mmol/L phosphate-EDTA buffer (pH 7.0) in the presence of IBMX (1 mmol/L) to inhibit cAMP degradation, followed by cooking at 100° C. for 7 minutes. This suspension was centrifuged, and the supernatant was used for ELISA assays with cAMP-specific antibodies (Stratagene, cat. no. 200020) using the manufacturer's instructions.

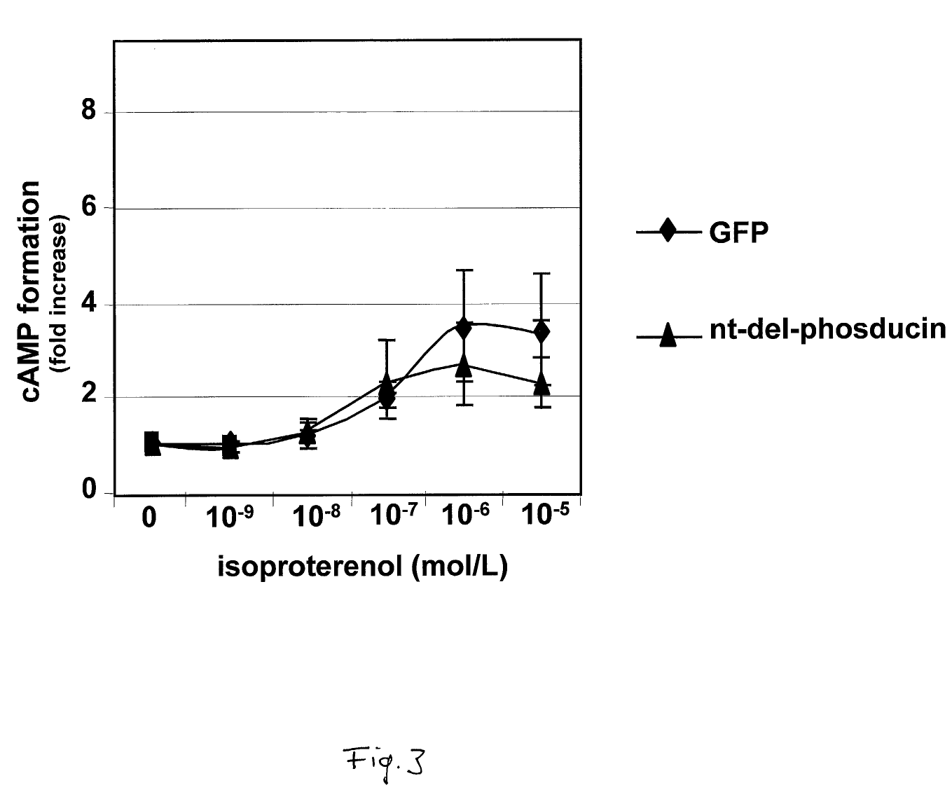

As a result, the β-adrenergic receptor agonist isoproterenol increased intracellular cAMP content in all groups (FIG. 3). In nt-del-phosducin-expressing cardiomyocytes, there was a trend towards decreased cAMP formation in response to isoproterenol, which did, however, not reach statistical significance (FIG. 3). Full-length phosducin has even been shown to significantly decrease maximal βAR-dependent adenylyl cyclase stimulability compared to controls in different cell types and tissues (Bauer et al. (1992) Nature. 358, 73-76; Schulz et al. J. Biol. Chem. (1996) 271, 22546-22551. The slight difference between the N-terminal truncated and full-length phosducin is most probably due to a higher Gβγ-binding capacity of the N-terminal truncated variant, which might explain different net effects on βAR-dependent cAMP accumulation.

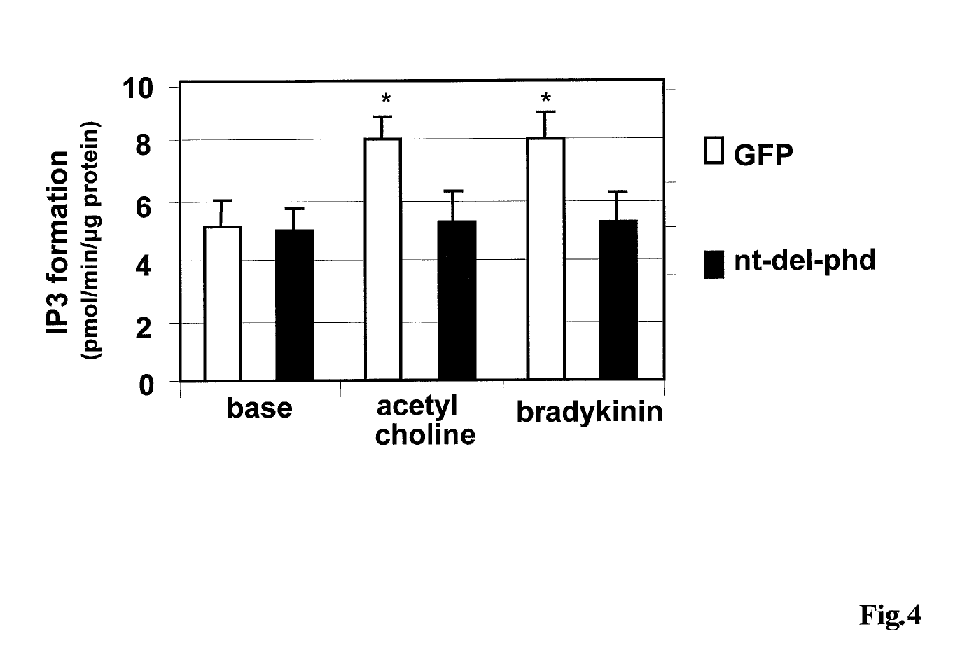

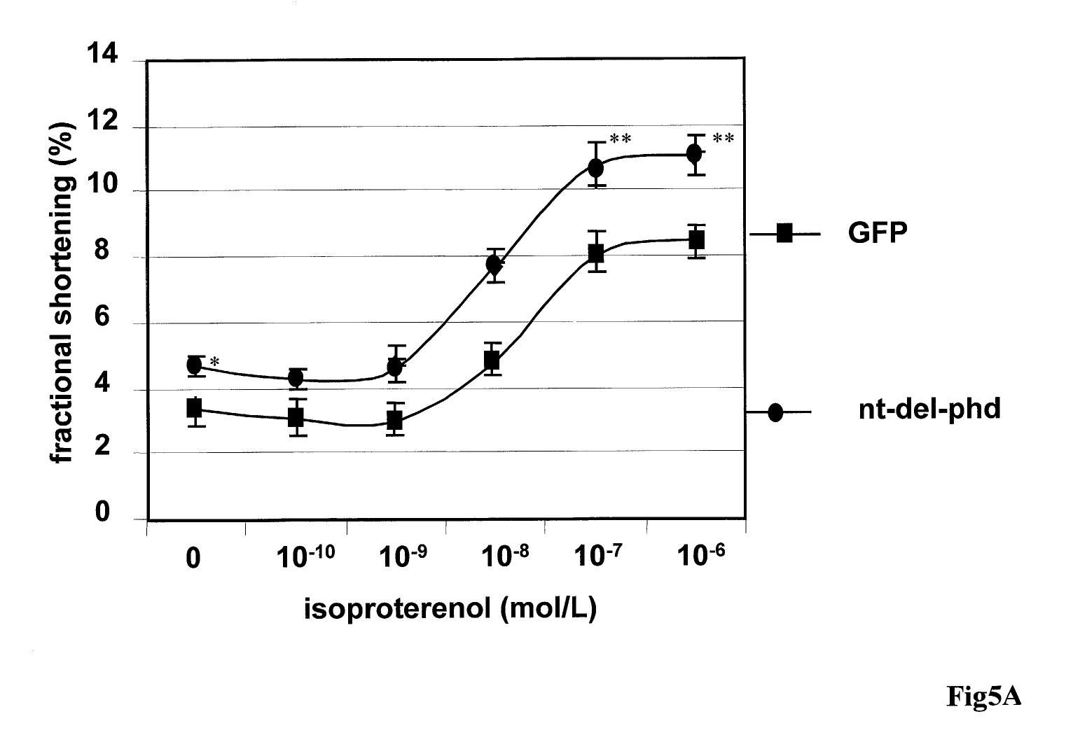

Inhibition of Gβγ-mediated effects after adenovirus delivery: The effect of the transgene on two Gβγ-dependent signalling pathways is demonstrated in FIG. 4 for the functional consequences of Gβγ inhibition on intracellular IP3 formation in response to both, bradykinin and acetylcholine. For IP3 assays, adenovirus-infected cardiomyocytes were stimulated with the respective agonists for 1 minute, and the reaction was stopped by adding perchloric acid (4%) and scratching the cells off. They were centrifuged at 2000×g, and then 10 pl of KOH (10 mol/L) was added. The solution was resuspended and centrifuged again, and the protein content of each sample was determined by the method of Bradford (1976) Anal. Biochem. 72, 248-254. The supernatant was used for an assay kit using ³H-inositol-(1,4,5)-trisphophate and a binding protein (Amersham, cat. no. TRK 1000) to measure IP3 formation, following the manufacturer's instructions. It is shown that intracellular IP3 formation is reduced in the presence of nt-del-phosducin, indicating that Gβγ-mediated effects were effectively inhibited by the transgene.

As a result of the experiments, it is shown that overexpression nt-del-phosducin results in a clear positive inotropic effect in both normal and failing cardiomyocytes after gene transfer ex vivo.

The direct functional significance of nt-del-phosducin overexpression on myocardial performance in the absence of tonic sympathoadrenal neural activation and mechanical loading is demonstrated based on the contractility of left ventricular myocytes isolated from normal or failing hearts after ex vivo gene transfer. Overexpression of nt-del-phosducin normal or failing hearts after ex vivo gene transfer. Overexpression of nt-del-phosducin enhances basal contraction and maximal contractility of both, normal or failing cardiomyocytes. Moreover, a clear leftward shift of the concentration-contractility curve occurred (FIG. 5).

### Example 4

### Functional Consequences of nt-del-Phosducin Binding to Gβγ in vivo

Model of Heart Failure: Medtronic pacemakers were implanted into New Zealand White rabbits (weight 3.6±0.3 Kg; from Harlan, Munich, Germany). Two days afterwards, rapid pacing was initiated at 320 beats/min. Under this protocol, a tachycardia-induced heart failure (HF) develops reproducibly over one week. Pacing was then continued at 360 beats/min, which predictably led to a further deterioriation of heart failure. The average contractility in failing hearts was 2200±320 mmHg/sec (vs. 4000±390 mmHg/sec in healthy controls; p<0.05), and LVEDP increased from 3.6±0.4 mmHg to 13.5±1.2 (p<0.05).

Adenoviral Gene Transfer To Rabbit Myocardium: After the first week of rapid pacing, all rabbits received catheter-based adenoviral gene transfer (5×10⁹ pfu) to the myocardium as described before (Weig et al. (2000) Circulation 101, 1578-1585). For the intervention, the rabbitswere anesthetized with fentanyl and propofol.

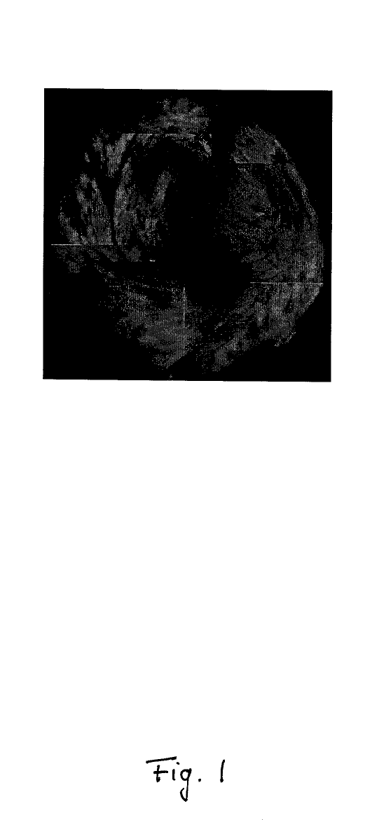

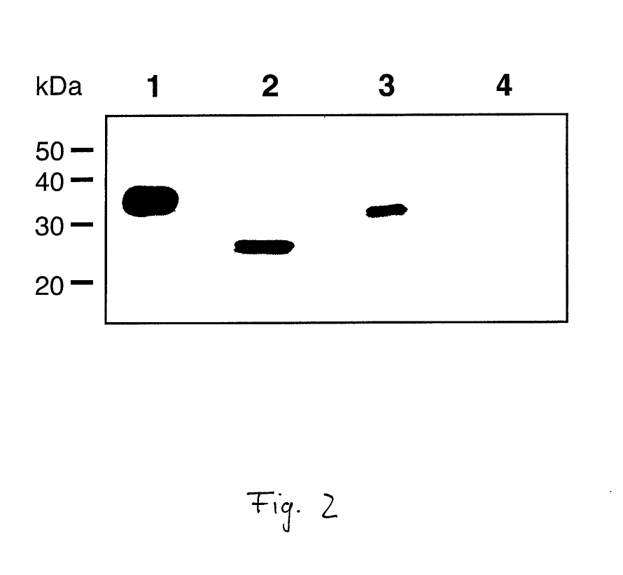

Overexpression of all transgenes was investigated by studying the co-expression of GFP in the hearts after in vivo gene transfer, since all transgenes were expressed bi-cistronically with GFP. To assess the efficacy of gene transfer in all hearts, transverse freeze-cut sections of myocardium for fresh histological analysis were obtained after the end of the experiments. The slices were stained with hematoxylin/eosin and Weigert by standard methods. Expression of GFP was examined in freeze-cut sections, using fluorescence microscopy. GFP co-expression could be determined throughout the left ventricle as examplarily shown in a macroscopic slice of a rabbit heart infected with Ad-nt-del-phosducin-GFP (FIG. 1). Western blotting documented that expression of nt-del-phosducin was detectable with a specific antibody, the transgene being 6 kD smaller than full-length phosducin (FIG. 2).

To assess the morphological change of the area of gene transfer, the hearts were perfused retrogradely with 4% paraformaldehyde, postfixed in Boun's solution and cut into 5-μm slices.

Myocardial Contractility Measurement by Echocardiography and Intraventricular Tip Catheterization: Left ventricular contractility was examined before the initiation of rapid pacing, before adenoviral gene transfer, and at the end of the protocol (two weeks after the start of pacing and one week after gene transfer). The rabbits were anesthetized as described before; ECG was monitored continuously.

For echocardiography, a 7.5 MHz probe was fixed on a tripod. Standard sections were recorded, which were well reproducible. For tip catheter measurements, a Millar 3F tip catheter connected to a differentiating device was placed in the left ventricle. After definition of basal contractility and left ventricular pressure, 200 μL of NaCl (0.9%) was injected as a negative control. Isoproterenol was infused at increasing doses. After a sufficient equilibration period, tip catheter measurements were carried out.

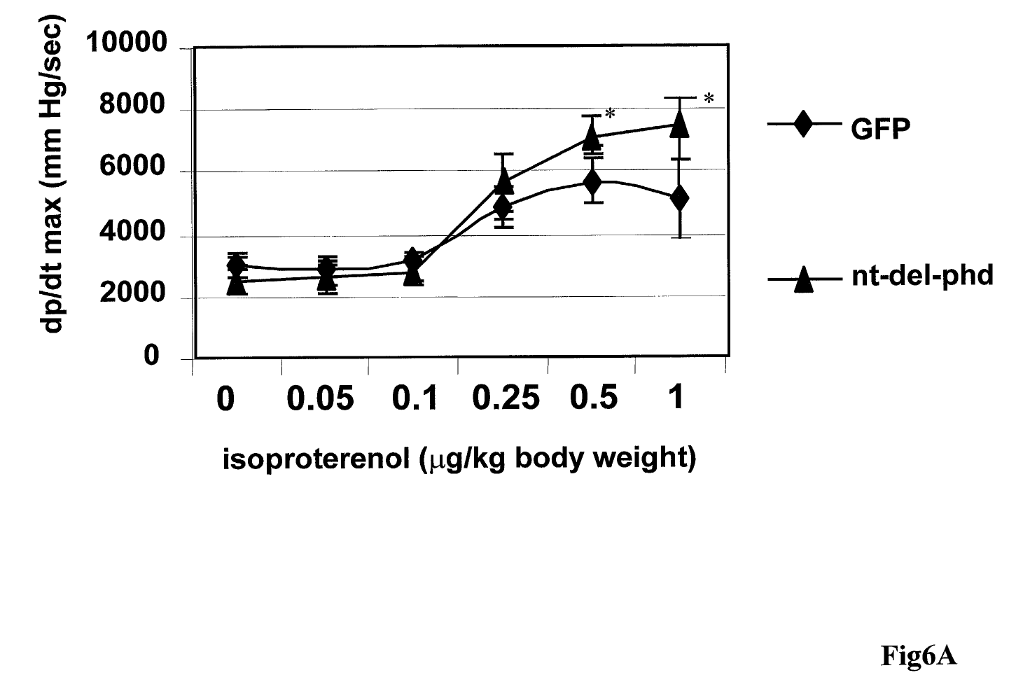

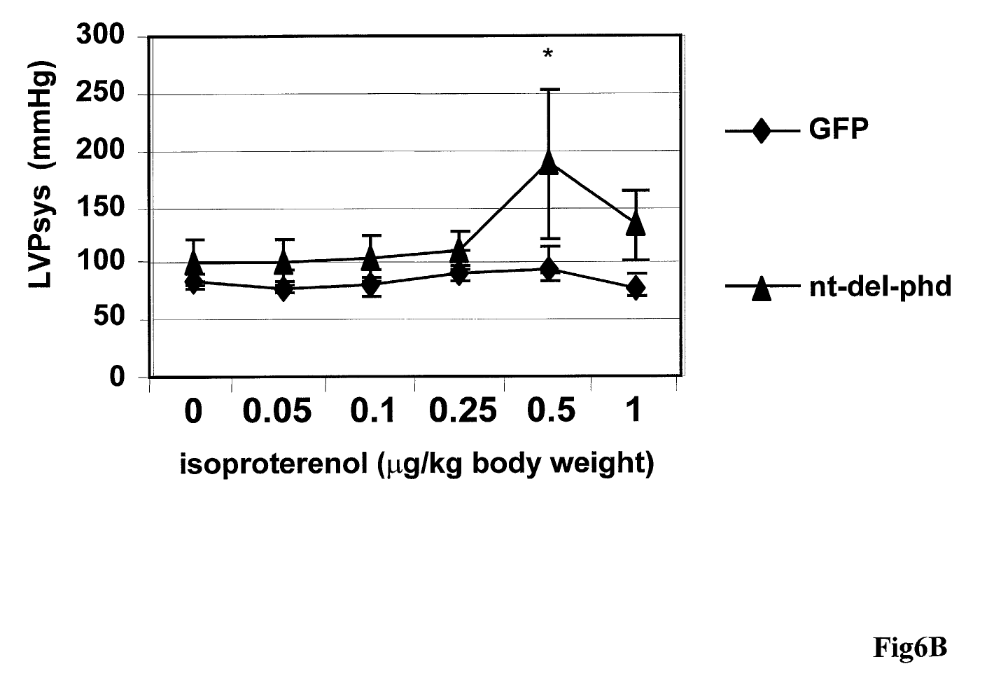

Improvement of LV dysfunction in pacing-induced heart failure after adenovirus delivery: Ad-GFP and Ad-nt-del-phosducin-GFP were directly delivered to rabbit hearts after 1 week of rapid pacing, and hemodynamic parameters were measured after another week of pacing at 360 bpm. For this purpose, serial echocardiography was carried out throughout the experiment. All experiments were terminated by an extensive tip catheterization. FIGS. 6A and 6B show the results from hemodynamic measurements of all groups.

In the nt-del-phosducin-expressing group, both the first derivative of LV pressure (dp/dt max) and the increase in systolic LV pressure in response to isoproterenol were significantly higher than in the Ad-GFP-infected control group.

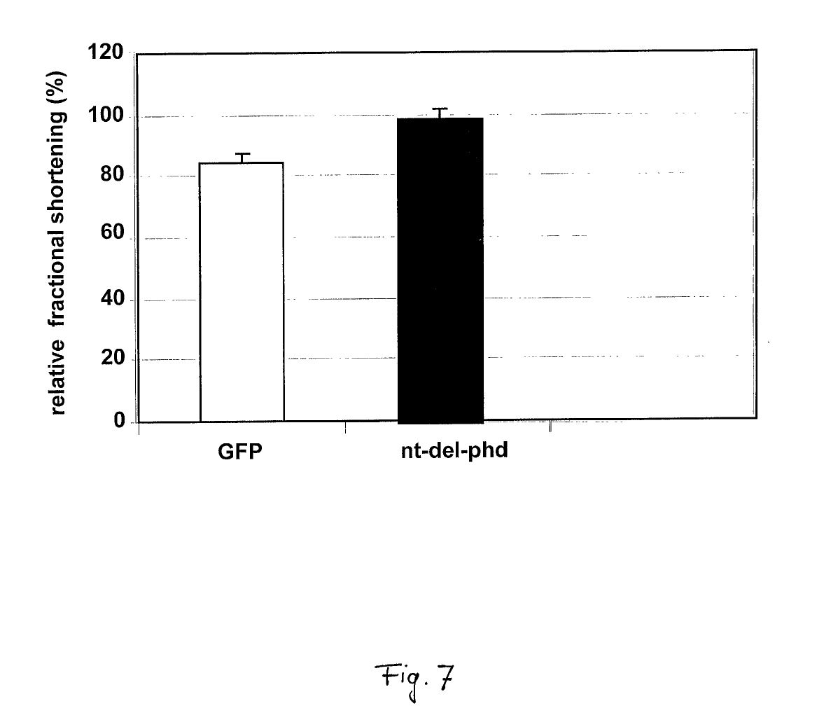

LV fractional shortening (FS) was followed by serial echocardiography, and the ratio of FS before gene transfer and at the end of the experiment was determined. In the nt-del-phosducin-expressing group, FS did not change during the second week of rapid pacing, wheres in the Ad-GFP-infected group, a clear decrease in FS occurred (FIG. 7).

As a result of the present exapmple, it is shown that cardiac function is clearly improved in rabbits with heart failure after in vivo gene transfer of the transgene. These results show that nt-del-phosducin exerts its positive effects by inhibition of those Gβγ-mediated pathways which are not linked to a resensitization of β-adrenergic receptors. Moreover, it is also shown that overexpression of nt-del-phosducin increased contractility and prevented further deterioriation of heart failure after in vivo gene transfer (FIG. 6).

In summary, it can be concluded that the augmentation in contractility induced by nt-del-phosducin is apparently independent of an increase in intracellular cAMP accumulation and therefore most probably unrelated to the resensitization of the receptors.

The beneficial effects of nt-del-phosducin on cardiac contractility in heart failure depend on their capacity to sequester Gβγ and consequently, to inhibit Gβγ-dependent pathways such as phospholipase C-β and phosphatidyltidylinositol (IP3) (Clapham et al. Nature. (1993) 365, 403-6) or mitogen-activated protein (MAP) kinase. MAP and PI3-kinase activities have recently been shown to be inhibited by activated Gβγ-βARK (Luttrell et al. Science (1999) 283, 655-61; Naga-Prasad et al. J. Biol. Chem. (2000) 275, 4693-4698).

### Example 5

### Inhibition of a Gβγ-Mediated Process in Cells: Phospholipase C Activity Assay with Phosducin-Transfected Cardiomycetes

For IP3 assays, cardiomyocytes infected with Ad-GFP or Ad-nt-del-phosducin-GFP (Example 1) were stimulated with 1 μmol/L bradykinin or 10 μmol/l acetyl choline, for 1 minute. In addition, basal levels were determined in the absence of agonists. Then the reaction was stopped by adding perchloric acid (4%) and scratching the cells off. They were centrifuged at 2000×g, and then 10 μl of KOH (10 mol/L) was added. The solution was resuspended and centrifuged again, and the protein content of each sample was determined by the method of Bradford²². The supernatant was used for an assay kit using ³H-inositol-(1,4,5)-trisphophate and a binding protein (Amersham, cat. no. TRK 1000) to measure IP3 formation, following the manufacturer's instructions. The results are shown in FIG. 4. Data represent means±SEM of 5 independent experiments. * p<0.05 nt-del-phosducin.

### Example 6

### Inhibition of a Gβγ-Mediated Process in Cells: Phospholipase C Activity Assay with Cardiomyocytes in the Presence of a Potential Inhibitor

IP3 assays were performed as described in Example 5 with the exception that non-transfected cardiomyocytes were used which were incubated for 10 minutes with 1 mM of the potential inhibitor of a Gβγ-mediated process prior to stimulating with 1 μmol/L bradykinin or 10 μmol/l acetylcholine.

### Example 7

### Inhibition of Gβγ-Stimulated Phospholipase C-β2 Activity by nt-del-phosducin using Purified Proteins

Phospholipase C activity was determined with a truncated phospholipase as described by Dietrich et al., (1994) Eur. J. Biochem. 219, 171-178. [³H]-Phosphatidyl 4,5-bisphosphate served as substrate. The concentration of Gβγ was 600 nM and that of nt-del-phosducin 100 μM. Activity is expressed as pmoles of inositol 1,4,5-triphosphate formed per minute.

### Example 8

### Inhibition of Gβγ-Stimulated Phospholipase C-β2 Activity by a Potential Inhibitor Using Purified Proteins

Phospholipase C activity was determined similarly as in Example 7 with the exception that nt-del-phosducin is replaced by 100 μM of a potential organic-chemical inhibitor.

### Example 9

### Determination of the Gβγ-Binding Capacity of Phosducin the Presence and Absence of a Potential Inhibitor

His-tagged nt-del-phosducin was produced according to Example 11.

Purified 6xHis-nt-del phosducin (250 pmol) is incubated with 130 pmol Gβγ purified from bovine brain (or from another animal's brain) and 500 pmol of a potential inhibotor in 200 μl phosphate-buffered saline containing 0.05% cholate (140 mM NaCl, 30 mM KCl, 6.5 mM Na₂HPO₄, pH 7.3). The proteins are then bound to 30 μl of Ni—NTA resin. The beads are washed in the same buffer in the presence of the potential inhibitor with intervening short centrifugations, and the bound Gβγ is detected by taking up the beads in SDS sample buffer followed by SDS polyacrylamide gel electrophoresis and Western blotting with antibodies against the γ-subunit (Signal transduction laboratories). Peroxidase-coupled antibodies are used to detect the blotting signal.

As a control, the same assay is conducted with the exception that the potential inhibitor is omitted, which gives maximal binding of Gβγ to the phosducin variant.

### Example 10

### Screening for a Compound Capable of Binding to Gβγ at a Binding Site of Phosducin

Wells of microtiter plates were coated with 300 ng of a βγ-complex for at least 4 h at 4° C. in 100 μl of 20 mM HEPES, 20 mM NaCl, 0.1 mM EDTA, pH 7.6 and 0.05% cholate (incubation buffer). The wells were washed several times with the same ice-cold buffer supplemented with 0.05% Tween 20 (wash buffer). After blocking with 3% bovine serum albumin in wash buffer, 10 μg of nt-del-phosducin (3 μM) and 100 μM of a potential antagonist were incubated in the wells at 4° C. for 2 h in 100 μl of incubation buffer plus 5 mM MgCl₂. The wells were then washed and blocked as above. Bound nt-del-phosducin was determined by addition of affinity-purified rabbit anti-phosducin antibodies for 1 h at room temperature. After incubation with peroxidase-coupled goat-anti-rabbit IgG, a color reaction was performed with o-phenylendiamine dihydrochloride (Sigma) and stopped with 50 μl of 3 M sulfuric acid. The absorption was measured at 490 nm.

As control, the same assay was performed with the exception that the potential antagonist was omitted.

### Example 11

### Cloning and Expression of nt-del-phosducin

His-tagged nt-del-phosducin was produced similarly as described by Bauer et al. (1992) Nature 358, 73-76 and according to standard procedures. Briefly, DNA coding for nt-del-phosducin was amplified from a plasmid containing a full-length phosducin gene by PCR using suitable primers. The PCR product was gel-purified and ligated into expression vector pQE30 (Qiagen). The obtained plasmid was transformed into E. coli strain BL21(DE3)pLysS. nt-del-phosducin expression was performed according to standard procedures. Cells were lysed in 50 mM Na-phosphate buffer (pH 7.4) by sonication. The lysate was centrifuged at 19,000 g for 30 minutes and the His-tagged protein was purified from the supernatant to 95% homogeneity by chromatography on Ni—NTA columns (Qiagen, Hilden, Germany).

1. A method of increasing the contractility of a heart, a heart muscle or cells of a heart muscle by administering an agent capable of binding to a phosducin binding site of Gβγ.

2. The method of claim 1, wherein the agent is a polypeptide.

3. The method of claim 2, wherein the polypeptide is an active phosducin, an active N-terminal truncated phosducin, or a function-conservative variant of an active phosducin or an active N-terminal truncated phosducin.

4. The method of claim 3, wherein the polypeptide is an N-terminal truncated phosducin or a function-conservative variant thereof.

5. The method of claim 4, wherein the polypeptide lacks at least 30 to 60 N-terminal amino acids of a natural phosducin.

6. The method of claim 2, wherein the polypeptide comprises amino acids 217 to 231 of a natural phosducin.

7. The method of claim 2, wherein the polypeptide comprises the amino acid sequence FLNEYGLL.

8. The method of claim 1, wherein the agent is an antibody against Gβγ.

9. The method of claim 1, wherein the agent is a nucleic acid which functions as an aptamer.

10. The method of claim 1, wherein the agent is a non-polypeptide drug.

11. The method of claim 1, wherein the agent binds to a binding site on Gβγ of an N-terminal truncated phosducin.

12. A method of increasing the contractility of a heart, a heart muscle or heart muscle cells by administering a vector encoding a polypeptide or a nucleic acid which are capable of binding to a phosducin binding site of Gβγ.

13. The method of claim 12, wherein the vector codes for a polypeptide as defined in claim 3.

14. A method of increasing the contractility of a heart, a heart muscle or heart muscle cells by administering a nucleic acid which inhibits expression of a Gβγ component by an anti-sense mechanism.

15. An active N-terminal truncated phosducin selected from the group of
(a) a polypeptide having the amino acid sequence of SEQ ID NO: 2 or function-conservative variants thereof;
(b) a polypeptide comprising the amino acid sequence of SEQ ID NO: 2, whereby the polypeptide lacks at least 50 N-terminal amino acids of a natural phosducin;
(c) a polypeptide having an amino acid sequence of least 80% identity as compared to the amino acid sequence of SEQ ID NO: 2.

16. A nucleic acid coding for an active N-terminal truncated phosducin selected from the group of
(a) a nucleic acid having the sequence of SEQ ID NO: 1 or any variant based on the degeneracy of the genetic code;
(b) a nucleic acid the complementary strand of which hybridizes under high stringency conditions with nucleic acids having the nucleic acid sequence of SEQ ID NO: 1;
(c) a nucleic acid having a sequence of at least 80% identity to the sequence of SEQ ID NO: 1.

17. A vector comprising a nucleotide sequence according to claim 16.

18. The vector according to claim 17, which is an adenoviral vector.

19. The vector according to claim 18, which is a gutless vector useful for somatic gene therapy.

20. An antibody or an aptamer capable of binding to a phosducin binding site of Gβγ.

21. A method of identifying a compound capable of binding to Gβγ at a binding site of phosducin, comprising the following steps:
(a) incubating a mixture comprising a predetermined concentration of Gβγ and a predetermined concentration of phosducin, an N-terminal truncated phosducin, or a function-conservative variant thereof under conditions which allow for binding of the phosducin, the N-terminal truncated phosducin, or the function conservative variant to Gβγ,
(b) incubating a mixture according to (a) under conditions according to (a) in the presence of a predetermined concentration of a test compound potentially capable of binding to Gβγ, and
(c) selecting a test compound providing a higher concentration of phosducin, of said N-terminal truncated phosducin or of said variant not bound to Gβγ in the mixture of step (b) than the mixture of step (a).

22. The method of claim 21, wherein step (b) is carried out by adding said test compound to the mixture of step (a).

23. A method of identifying a compound which increases the contractility of muscle cells, comprising the following steps:
(a) measuring the contractility of isolated muscle cells after stimulation, preferably with a β-adrenergic receptor agonist,
(b) measuring the contractility of isolated muscle cells according to (a), whereby said muscle cells are further exposed to a test compound potentially increasing the contractility of the muscle cells, and
(c) selecting a test compound which causes a higher contractility in step (b) than in step (a).

24. The method of claim 23, wherein the muscle cells are heart muscle cells.

25. Method of identifying a compound which increases the contractility of a heart muscle, wherein the method of claim 23 is carried out with a compound selected in step (c) of the method of claim 21.

26. Compound obtained or obtainable according to the method of one of claims 21 to 25.

27. Method of increasing the contractility of muscle cells in a heart, comprising the administration of phosducin, a function-conservative variant thereof or a nucleic acid coding therefor.

28. Method for identifying a compound which inhibits Gβγ-mediated processes, comprising the following steps:
(i) incubating a mixture comprising Gβγ and a downstream component of a Gβγ-mediated process in pre-defined concentrations, whereby said component is controlled by a Gβγ-mediated process in the mixture,
(ii) incubating, under conditions as in (i), a mixture comprising Gβγ, said downstream component of a Gβγ-mediated process and a test compound which potentially inhibits Gβγ-mediated processes and
(iii) selecting a test compound which inhibits Gβγ in the Gβγ-mediated process.

29. The method of claim 28, wherein said component is phospholipase Cβ, said mixtures further contain phosphatidylinositol and said inhibition is determined via the enzymatic activity of phospholipase Cβ.

30. Method of identifying a compound which inhibits Gβγ-mediated processes in cells, comprising the following steps:
(i) incubating cells with an agonist of a G-protein-coupled receptor and measuring a signal due to the amount or activity of a component of a Gβγ-mediated process,
(ii) incubating cells, under conditions as in (i), with said agonist and a test compound which potentially inhibits Gβγ-mediated processes and measuring said signal due to the amount or activity of said component of said βγ-mediated process, and
(iii) selecting a test compound which results in a lower amount or activity of said component in step (ii) than in step (i).

31. The method of claim 30, wherein said component is phospholipase Cβ the activity of which is determined via its enzymatic activity.

32. The method of claim 30, wherein said component is the bradykinin B1 receptor.

33. The method of claim 30, wherein said cells are muscle cells, preferably of a heart muscle.

34. A method of identifying a compound which increases the contractility of muscle cells, comprising the following steps:
(i) obtaining a set of atomic coordinates defining the three-dimensional structure of the binding site of phosducin to a Gβγ protein complex;
(ii) selecting a test compound by performing rational drug design with the atomic coordinates obtained in step (i), wherein said selecting is performed in conjunction with computer modeling;
(iii) contacting the test compound with a muscle cell; and
(iv) measuring the contractility under predetermined conditions under which the muscle cell has a predetermined contractility;
wherein a test compound is identified as a compound that increases contractility when there is a higher contractility in the presence of the test compound relative to in its absence.

35. A method of identifying a compound for use as an inhibitor of Gβγ-mediated processes comprising:
(i) obtaining a set of atomic coordinates defining the three-dimensional structure of the binding site of phosducin to a Gβγ protein complex;
(ii) selecting a test compound by performing rational drug design with the atomic coordinates obtained in step (i), wherein said selecting is performed in conjunction with computer modeling;
(iii) contacting the test compound with Gβγ in a mixture allowing measurement of a Gβγ-mediated process; and p1 (iv) measuring a Gβγ-mediated process;
wherein a test compound is identified as a compound that inhibits Gβγ-mediated processes when there is a decrease in the activity of the Gβγ-mediated process in the presence of the test compound relative to in its absence.

36. A screening kit for identifying compounds capable of binding to Gβγ, comprising an active N-terminal truncated phosducin which may be labeled and a Gβγ.


SEQUENCE LISTING (2 sequences)

SEQ ID NO: 1 - DNA, 582 bp, Homo sapiens
atgtcttctcctcagagtagagatgacaaagactcaaaagaaagattcagcagaaagatgagcgttcaagaatatgaactaatccacaaagacaaagaagatgaaaattgccttcgtaaataccgcagacagtgtatgcaggatatgcaccagaagctgagttttgggcctagatatgggtttgtgtatgagctggaatctggggagcaattcctggaaaccattgaaaaggaacagaaaatcaccactatcgttgttcatatttatgaagatggtattaagggctgtgatgctctaaacagtagcttgatatgccttgcagccgaataccctatggtcaagttttgtaaaataaaggcttctaatacaggtgccggagaccgcttttcctcagatgtactccccacgctgcttgtctacaaaggtggggaactcctaagcaatttcattagtgttactgaacagctggctgaagagttttttactggggatgtggagtctttcctaaatgaatatgggttattacctgaaaaggagatgcatgtcctagagcagagcaacatggaagaggatatggaataa

SEQ ID NO: 2 - protein, 193 aa, Homo sapiens
MSSPQSRDDKDSKERFSRKMSVQEYELIHKDKEDENCLRKYRRQCMQDMHQKLSFGPRYGFVYELESGEQFLETIEKEQKITTIVVHIYEDGIKGCDALNSSLICLAAEYPMVKFCKIKASNTGAGDRFSSDVLPTLLVYKGGELLSNFISVTEQLAEEFFTGDVESFLNEYGLLPEKEMHVLEQSNMEEDME